(19) United States
(12) Patent Application Publication
(10) Pub. No.: US 2009/0088463 A1
(43) Pub. Date: Apr. 2, 2009

(54) Pyrazoles Useful in the Treatment of Inflammation

(76) Inventors: Benjamin Pelcman, Solna, (SE); Andrei Sanin, Solna (SE); Peter Nilsson, Solna (SE)

Correspondence Address: MORGAN LEWIS & BOCKIUS LLP, 1111 PENNSYLVANIA AVENUE NW, WASHINGTON, DC, 20004, US

(21) Appl. No.: 12/084,426

(22) PCT Filed: Oct. 30, 2006
(86) PCT No.: PCT/GB2006/004042

§ 371 Date: Oct. 1, 2008

Related U.S. Application Data
(60) Provisional application No. 60/731,928, filed on Nov. 1, 2005.

(51) Int. Cl. A61K 31/415; C07D 231/14; A61P 29/00

(52) U.S. Cl. 514/406; 548/374.1

(57) ABSTRACT

There is provided compounds of formula (I), wherein X¹ to X⁵ have meanings given in the description, and pharmaceutically-acceptable salts thereof, which compounds are useful in the treatment of diseases in which inhibition of the activity of a lipoxygenase (e.g. 15-lipoxygenase) is desired and/or required, and particularly in the treatment of inflammation.

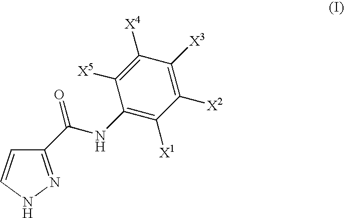

### FIELD OF THE INVENTION

The invention relates to novel pharmaceutically-useful compounds. The invention further relates to compounds that are useful in the inhibition of the activity of 15-lipoxygenase and thus in the treatment of inflammatory diseases and of inflammation generally. The invention also relates to the use of such compounds as medicaments, to pharmaceutical compositions containing them, and to synthetic routes for their production.

### BACKGROUND OF THE INVENTION

There are many diseases/disorders that are inflammatory in their nature. One of the major problems associated with existing treatments of inflammatory conditions is a lack of efficacy and/or the prevalence of side effects (real or perceived).

Asthma is a chronic inflammatory disease affecting 6% to 8% of the adult population of the industrialized world. In children, the incidence is even higher, being close to 10% in most countries. Asthma is the most common cause of hospitalization for children under the age of fifteen.

Treatment regimens for asthma are based on the severity of the condition. Mild cases are either untreated or are only treated with inhaled β-agonists. Patients with more severe asthma are typically treated with anti-inflammatory compounds on a regular basis.

There is a considerable under-treatment of asthma, which is due at least in part to perceived risks with existing maintenance therapy (mainly inhaled corticosteroids). These include risks of growth retardation in children and loss of bone mineral density, resulting in unnecessary morbidity and mortality. As an alternative to steroids, leukotriene receptor antagonists (LTRas) have been developed. These drugs may be given orally, but are considerably less efficacious than inhaled steroids and usually do not control airway inflammation satisfactorily.

This combination of factors has led to at least 50% of all asthma patients being inadequately treated.

A similar pattern of under-treatment exists in relation to allergic disorders, where drugs are available to treat a number of common conditions but are underused in view of apparent side effects. Rhinitis, conjunctivitis and dermatitis may have an allergic component, but may also arise in the absence of underlying allergy. Indeed, non-allergic conditions of this class are in many cases more difficult to treat.

Chronic obstructive pulmonary disease (COPD) is a common disease affecting 6% to 9% of the world population. The disease is potentially lethal, and the morbidity and mortality from the condition is considerable. At present, there is no known pharmacological treatment capable of changing the course of COPD.

Other inflammatory disorders which may be mentioned include:
(a) pulmonary fibrosis (this is less common than COPD, but is a serious disorder with a very bad prognosis. No curative treatment exists);
(b) inflammatory bowel disease (a group of disorders with a high morbidity rate. Today only symptomatic treatment of such disorders is available); and
(c) rheumatoid arthritis and osteoarthritis (common disabling inflammatory disorders of the joints. There are currently no curative, and only moderately effective symptomatic, treatments available for the management of such conditions).

Inflammation is also a common cause of pain. Inflammatory pain may arise for numerous reasons, such as infection, surgery or other trauma. Moreover, several malignancies are known to have inflammatory components adding to the symptomotology of the patients.

Thus, a new and/or alternative anti-inflammatory treatment would be of benefit to all of the above-mentioned patient groups. In particular, there is a real and substantial unmet clinical need for an effective anti-inflammatory drug capable of treating inflammatory disorders, such as asthma, with no real or perceived side effects.

The mammalian lipoxygenases are a family of structurally-related enzymes, which catalyze the oxygenation of arachidonic acid. Three types of human lipoxygenases are known, which catalyze the insertion of molecular oxygen into arachidonic acid at carbon positions 5, 12 and 15. The enzymes are thus named 5-, 12- and 15-lipoxygenase, respectively.

Arachidonic acid metabolites that are formed following the action of lipoxygenases are known to have pronounced pathophysiological activity including pro-inflammatory effects.

For example, the primary product of the action of 5-lipoxygenase on arachidonic acid is further converted by a number of enzymes to a variety of physiologically and pathophysiologically important metabolites. The most important of these, the leukotrienes, are strong bronchoconstrictors. Huge efforts have been devoted towards the development of drugs that inhibit the action of these metabolites as well as the biological processes that form them. Drugs that have been developed to this end include 5-lipoxygenase inhibitors, inhibitors of FLAP (Five Lipoxygenase Activating Protein) and, as mentioned previously, leukotriene receptor antagonists (LTRas).

Another class of enzymes that metabolize arachidonic acid are the cyclooxygenases. Arachidonic acid metabolites that are produced by this process include prostaglandins, thromboxanes and prostacyclin, all of which possess physiological or pathophysiological activity. In particular, the prostaglandin PGE₂ is a strong pro-inflammatory mediator, which also induces fever and pain. Consequently, a number of drugs have been developed to inhibit the formation of PGE₂, including “NSAIDs” (non-steroidal antiinflammatory drugs) and “coxibs” (selective cyclooxygenase-2 inhibitors). These classes of compounds act predominantly by way of inhibition of one or several cyclooxygenases.

Thus, in general, agents that are capable of blocking the formation of arachidonic acid metabolites are likely to be of benefit in the treatment of inflation.

### PRIOR ART

International patent application WO 2004/080999 discloses various 1(N)-substituted-3-amidopyrazoles for use in the treatment of inflammation, some of which are synthesised via 1(N-unsubstituted-3-amidopyrazole intermediates. Accordingly, there is no suggestion in this document that such 1-(N)-unsubstituted-3-amidopyrazoles have any use in the treatment of inflammation.

International patent application WO 2006/032852 discloses various 1(N)-substituted-3-amidopyrazoles that contain at least one substituent on the pyrazole ring for use in the treatment of inflammation, some of which are synthesised via 1(N)-unsubstituted-3-amidopyrazole intermediates. However, there is no suggestion in this document of 1-(N)-unsubstituted-3-amidopyrazoles that are unsubstituted in the 4- and 5-position of the pyrazole ring.

International patent application WO 2006/032851 discloses various 3-amidopyrazoles for the treatment of inflammation, wherein the amido group is substituted with a bicyclic heterocyclic group. However, there is no disclosure or suggestion of corresponding 3-amidopyrazoles in which the amido group is substituted by a monocyclic aromatic group.

Japanese patent application JP 2002241273 discloses compounds for use in the treatment of hyperlipemia by inhibition of FXR transcription activity. The use of such compounds in the treatment of inflammation is not suggested.

Pyrazole-based compounds have been disclosed in several publications. For example, international patent application WO 2003/016304 discloses various heterocycles, including pyrazoles, for use in controlling invertebrate pests, international patent application WO 01/57024 discloses various pyrazoles that are useful in blockading voltage-dependent sodium channels; international applications WO 03/020217 and WO 01158869, and US Patent No. 2004/0192667 disclose various nitrogen-containing heterocycles, including pyrazoles, that are useful as cannabinoid receptor modulators; international patent application WO 99/20294 discloses pyrazoles that are useful in the treatment of cystic fibrosis; international application WO 2005/007625 discloses anti-tuberculosis compounds that include pyrazoles; U.S. Pat. No. 2003/0091116 and international patent applications WO 01/19798, WO 99/32454 and WO 2004/055815 disclose inter alia pyrazoles that may be useful as Factor Xa inhibitors; and WO 01/21160 discloses antiviral compounds that include pyrazoles. There is no disclosure in any of these documents of 1(N-unsubstituted-3-amidopyrazoles for use in treating inflammation and/or as inhibitors of lipoxygenases.

International patent application WO 2005/016877 discloses 5-substituted pyrazoles that may be useful in the inhibition of 11β-hydroxysteroid dehydrogenase-1 (and therefore useful in the treatment of inter alia diabetes). There is no specific disclosure in this document of pyrazoles that are substituted in the 3-position with an aromatic amido group.

Vertuani et al., Journal of Pharmaceutical Sciences, Vol. 74, No. 9 (1985) discloses that various 3-methyl-N-phenyl-1H-pyrazol-5-ylcarboxamides, possess anti-inflamatory and analgesic activities. However, there is no disclosure or suggestion in this document of 1-(N)-unsubstituted-3-amidopyrazoles that do not contain any further substituents on the pyrazole ring.

International patent applications WO 03/037274 and US patent application publication No. 2005/0049237 disclose various pyrazoles that may be useful in treating inflammatory pain, which mechanism works by blocking sodium channels. This document relates primarily to pyrazoles that are 1(N)-substituted and/or to pyrazoles that are substituted by an amido group in the 4-position.

International patent application WO 03/068767 also relates to inter alia pyrazole-containing compounds that may be useful in treating inflammatory pain by opening potassium ion channels. However, this document relates only to pyrimidinyl amido compounds.

International patent application WO 97/19062 discloses various pyrazoles for the treatment of skin related diseases and further mentions the use of such compounds in the treatment of various inflammatory diseases. However, this document does not mention or suggest 3-amido pyrazoles that are substituted at the 4- and/or 5-position of the pyrazole ring with a halo or trifluoromethyl group.

International patent application WO 2004/096795 discloses various heterocycles, including pyrazoles, as inhibitors of protein tyrosine kinases and international patent application WO 01/55115 discloses various aromatic amides that may be useful as activators of caspases and inducers of apoptosis. Accordingly, the compounds disclosed in these documents may be useful in the treatment of inter alia cancer. There is no disclosure or suggestion in either of these documents of the use of such compounds as inhibitors of lipoxygenases.

Finally, international application WO 99/25695 relates to 1(N)-substituted-5-arylpyrazole compounds that may be useful in treating inflammatory pain by cyclooxegenase inhibition. There is no disclosure in this document of 1(N)-unsubstituted-3-amidopyrazoles for use in treating inflammation and/or as inhibitors of lipoxygenases.

### DISCLOSURE OF THE INVENTION

According to the invention there is provided a compound of formula I,

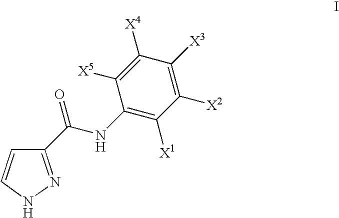

wherein
X¹ represents halo, —CN, —NO₂, —R¹ or —OR²;
X² to X⁵ independently represent H, halo, —CN, —NO₂, —R¹ or —OR², wherein at least one of X³ or X⁴ is other than H;.
R² represents, on each occasion when mentioned above, H or R¹; and
R¹ represents, on each occasion when mentioned herein, C₁₋₆ all optionally substituted by one or more substituents selected from F, Cl, —OCH₃, —OCH₂CH₃, —OCHF₂ and —OCF₃;
or a pharmaceutically acceptable salt thereof;
provided that when X² represents H:
(a) X¹ represents —CN and X³ and X⁵ both represent H, then X⁴ does not represent Cl;
(b) X¹ represents Cl and X⁴ and X⁵ both represent H, then X³ does not represent F;
(c) X¹ represents F and X³ and X⁵ both represent H, then X⁴ does not represent —CF₃;
(d) X¹ represents Br and X⁴ and X⁵ both represent H, then X³ does not represent —OCF₃;
(e) X¹ represents —OH, X³ represents H and X⁴ represents t-Bu, then X⁵ does not represent —OH; and
(f) X¹ represents —CH₃ and X⁴ and X⁵ both represent H, then X³ does not represent —CH₃,
   which compounds and salts are referred to hereinafter as “the compounds of the invention”.

Pharmaceutically-acceptable salts include acid addition salts and base addition salts. Such salts may be formed by conventional means, for example by reaction of a free acid or a free base form of a compound of formula I with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of the invention in the form of a salt with another counter-ion, for example using a suitable ion exchange resin.

Compounds of the invention may contain double bonds and may thus exist as E (entgegen) and Z (zusammen) geometric isomers about each individual double bond. All such isomers and mixtures thereof are included within the scope of the invention.

Compounds of the invention may also exhibit tautomerism All tautomeric forms and mixtures thereof are included within the scope of the invention.

Compounds of the invention may also contain one or more asymmetric carbon atoms and may therefore exhibit optical and/or diastereoisomerism. Diastereoisomers may be separated using conventional techniques, e.g. chromatography or fractional crystallisation. The various stereoisomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which will not cause racemisation or epimerisation (i.e. a ‘chiral pool’ method), by reaction of the appropriate starting material with a ‘chiral auxiliary’ which can subsequently be removed at a suitable stage, by derivatisation (i.e. a resolution, including a dynamic resolution), for example with a homochiral acid followed by separation of the diastereomeric derivatives by conventional means such as chromatography, or by reaction with an appropriate chiral reagent or chiral catalyst all under conditions known to the skilled person All stereoisomers and mixtures thereof are included within the scope of the invention.

Unless otherwise specified, C_{1-q} alkyl (where q is the upper limit of the range), defined herein may be straight-chain or, when there is a sufficient number (i.e. a minimum of three) of carbon atoms, be branched-chain, and/or cyclic (so forming, in the case of alkyl, a C_{3-q} cycloakyl group). Further, when there is a sufficient number (i.e. a minimum of four) of carbon atoms, such groups may also be part cyclic. Further, unless otherwise specified, such alkyl groups may also be saturated or, when there is a sufficient number (i.e. a minimum of two) of carbon atoms and unless otherwise specified, be unsaturated (forming, for example, a C_{2-q} alkenyl or a C_{2-q} alkynyl group).

The term “halo”, when used herein, includes fluoro, chloro, bromo and iodo.

For the avoidance of doubt, in cases in which the identity of two or more substituents in a compound of the invention may be the same, the actual identities of the respective substituents are not in any way interdependent. For example, in the situation in which X¹ and X² both represent R¹, in which R¹ is a C₁₋₆ alkyl group, the respective alkyl groups may be the same or different.

Compounds of the invention that may be mentioned include those in which:

when X¹ and/or X⁵ represent —OR², then R² represents R¹; and
when X⁴ represents R¹, then R¹ represents C₁₋₃ alkyl or C₅₋₆ alkyl optionally substituted by one or more substituents selected from F, Cl, —OCH₃, —OCH₂CH₃, —OCHF₂ and —OCF₃, particularly when X¹ and/or X⁵ represent OH and/or X³ represents H.

Further compounds of the invention that may be mentioned include those in which:

R¹ and/or R² independently represent C₁₋₆ alkyl (e.g. C₁₋₄ alkyl) optionally substituted by one or more substituents selected from F, —OCH₃, —OCH₂CH₃, —OCHF₂ and —OCF₃.

Preferred compounds of the invention include those in which:

X² to X⁴ independently represent I or, preferably, —R¹, —OR² or, more preferably, H, F, Cl or Br.

Preferred compounds of the invention include those in which:

X¹ represents —CN, —OR² or, more preferably, Br, Cl, F or R¹;
X² represents F, Cl or, more preferably, H;
X³ represents Br, I or, preferably, H, Cl, F, R¹, or —OR²;
X⁴ represents Cl, F, R¹, or more preferably H;
R¹ and R² independently represent H or, preferably, C₁₋₂ alkyl (e.g. methyl) optionally substituted by one or more F substituent (so forming, for example, a trifluoromethyl group);
R¹ and R² independently represent H or, preferably, methyl, ethyl, difluoromethyl, trifluoromethyl or 1,1,1-trifluoroethyl;
X⁵ represents H.

Particularly preferred compounds of the invention include those of the examples described hereinafter.

Compounds of the invention may be made in accordance with techniques that are well known to those skilled in the art, for example as described hereinafter.

According to a further aspect of the invention there is provided a process for the preparation of a compound of formula I, which process comprises:

(i) reaction of pyrazole-3-carboxylic acid, or a N-protected and/or O-protected derivative thereof, with a compound of formula II,

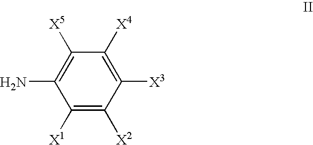

wherein X¹ to X⁵ are as hereinbefore defined, under coupling conditions, for example at around room temperature or above (e.g. up to 40-180° C.), optionally in the presence of a suitable base (e.g. sodium hydride, sodium bicarbonate, potassium carbonate, pyrrolidinopyridine, pyridine, triethylamine, tributylamine, trimethylamine, dimethylaminopyridine, diisopropylamine, diisopropylethylamine, 1,8-diazabicyclo[5.4.0]undec-7-ene, sodium hydroxide, N-ethyldiisopropylamine, N-(methylpolystyrene)-4-(methylamino)pyridine, butyllithium (e.g. n-, s- or t-butyllithium) or mixtures thereof), an appropriate solvent (e.g. tetrahydrofuran, pyridine, toluene, dichloromethane, chloroform, acetonitrile, dimethylformamide, dimethylsulfoxide, water or triethylamine) and a suitable coupling agent (e.g. 1,1′-carbonyldiimidazole, N,N′-dicyclohexylcarbodiimide, 1 (3-dimethylamino-propyl)-3-ethylcarbodiimide (or hydrochloride thereof), N,N′-disuccinimidyl carbonate, benzothiazol-1-yloxytris(dimethylamino) phosphonium hexafluoro-phosphate, 2-(1H-benzotriazol-1-3)-1,1,3,3-tetramethyluronium hexafluorophos-phate, benzotriazol-1-yloxytrispyrrolidinophosphonium hexafluorophosphate, bromo-tris-pyrrolidinophosphonium hexafluorophosphate, 2-(1H-benzotriazol-1-yl)-1,1,3,3-tetramethyluronium tetrafluorocarbonate, 1-cyclohexylcarbodiimide-3-propyloxymethyl polystyrene, O-(7-azabenzotriazol-1-yl)-N,N,N′,N′-tetramethyluronium hexafluorophosphate or O-benzotriazol-1-yl-N,N,N′,N′-tetramethyluronium tetrafluoroborate). Alternatively, pyrazole-3-carboxylic acid may first be activated by treatment with a suitable reagent (e.g. oxalyl chloride, thionyl chloride, etc) optionally in the presence of an appropriate solvent (e.g. dichloromethane, TEHF, toluene or benzene) and a suitable catalyst (e.g. DMF), resulting in the formation of the respective acyl chloride. This activated intermediate may then be reacted with a compound of formula II under standard conditions, such as those described above. The skilled person will appreciate that when compounds of formula II are liquid in nature, they may serve as both solvent and reactant in this reaction. Alternative methods of performing this step include reaction of an O-protected derivative (e.g. an ethyl ester) of pyrazole-3-carboxylic acid with a compound of formula II, which latter compound may first be treated with an appropriate reagent (e.g. trimethylaluminium), for example in an inert atmosphere and in the presence of a suitable solvent (e.g. dichloromethane);
(ii) reaction of pyrazole, or a N-protected derivative thereof, with a suitable base, (or a mixtures of bases), such as potassium bis(timethylsilyl)amide, sodium bis(trimethylsilyl)amide, sodium hydride, potassium tert-butoxide or an organolithium base, such as n-BuLi, s-BuLi, t-BuLi lithium diisopropylamide or lithium 2,2,6,6-tetramethylpiperidine (which organolithium base is optionally in the presence of an additive (for example, a lithium co-ordinating agent such as an ether (e.g. dimethoxyethane) or an amine (e.g. tetramethylethylenediamine (TMEDA), (−)sparteine or 1,3-dimethyl-3,4,5,6-tetrahydro-2(1H)-pyrimidinone (DNU) and the like)), followed by reaction with a compound of formula III,

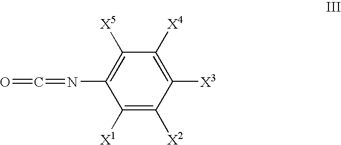

wherein X¹ to X⁵ are as hereinbefore defined, followed by quenching with a suitable proton source (e.g. water or aqueous, saturated NH₄Cl solution). The skilled person will appreciate that pyrazole may need to be protected at the nitrogen atom of the pyrazole ring system, preferably with a protective group that is also a directing metallation group (such as a benzenesulfonyl group). The reaction may be performed in the presence of a suitable solvent, such as a polar aprotic solvent (e.g. tetrahydrofuran or diethyl ether), at sub-ambient temperatures (e.g. 0° C. to −78° C.) under an inert atmosphere followed (as appropriate) by deprotection of the N-protective group under standard conditions (e.g. when a benzenesulfonyl group is employed, by hydrolysis). Furthermore, the skilled person will appreciate that the amido group will be introduced a to one of the pyrazole nitrogen atoms, and thus there are two alternative positions;
(iii) reaction of dipyrazolo[1,5-a;1′,5′-d]pyrazine-4,9-dione, with a compound of formula II as hereinbefore defined for example under coupling conditions such as those described hereinbefore in respect of process step (i) above. Preferred conditions include reaction in the presence of base and solvent but no coupling reagent In this case, compound of formula II may also be employed in excess; or
(iv) reaction of pyrazole-3-carboxamide or a N-protected (e.g. at the pyrazole nitrogen) derivative thereof, with a compound of formula IV,

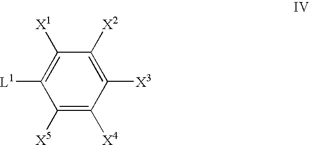

wherein L¹ represents a suitable leaving group, such as halo (e.g. chloro, bromo and iodo), —OSO₂CF₃, —B(OH)₂, —Sn(R^{z})₃ (in which each R^{z} independently represents C₁₋₆ alkyl (e.g. methyl or butyl)), —Pb(CC(O)CH₃)₃, —Bi(W)₂, —Bi(W)₂(OC(O)CH₃)₂, —Bi(W)₂(OC(O)CF₃)₂ or —I(W)(BF₄), and W represents an optionally substituted aryl or heteroaryl group and, preferably, W represents the phenyl ring of the compound of formula II as hereinbefore defined, and X¹ to X⁵ are as hereinbefore defined, for example in the presence of a catalyst containing, preferably, Pd or Cu, and a base, such as potassium or sodium hydroxide, potassium carbonate, potassium tert-butoxide and lithium N,N-diisopropylamide. Catalysts that may be mentioned include Pd₂(dba)₃ (tris(dibenzylideneacetone)dipalladium(0)), bases that may be mentioned include cesium carbonate, ligands that may be mentioned include 2,2′-bis(diphenylphosphino)-1,1′-binaphthyl and solvents that may be employed include toluene. Such reactions may be performed at elevated temperature (e.g. at about 90° C.) under an inert (e.g. argon) atmosphere.

Dipyrazolo[1′,5-a;1′,5′-d]pyrazine-4,9-dione may be prepared from pyrazole-3-carboxylic acid under dimerising conditions, for example in the presence of thionyl chloride (optionally in the presence of a suitable solvent and catalyst, such as one hereinbefore defined in respect of process step (i)) at reflux. Other dimerising reagents include or oxalyl chloride or carbodiimides, such as carbonyldiimidazole, 1,3-dicyclohexylcarbodiimide or 1-(3-dimethylaminopropyl)-3-ethylcarbodiimide (EDCI, or hydrochloride thereof) optionally in the presence of a suitable base (e.g. 4-dimethylaminopyridine).

Pyrazole-3-carboxylic acid, dipyrazolo[1,5-a;1′,5′-d]pyrazine-4,9-dione, pyrazole-3-carboxamide and compounds of formulae II, III and IV are either commercially available, are known in the literature, or may be obtained either by analogy with the processes described herein, or by conventional synthetic procedures, in accordance with standard techniques, from available starting materials using appropriate reagents and reaction conditions. In this respect, the skilled person may refer to inter alia “Comprehensive Organic Synthesis” by B. M. Trost and I. Fleming, Pergamon Press, 1991.

The substituents X¹ to X⁵ as hereinbefore defined may be modified one or more times, after or during the processes described above for preparation of compounds of formula I by way of methods that are well known to those skilled in the art. Examples of such methods include substitutions, reductions, oxidations, alkylations, acylations, hydrolyses, esterifications, and etherifications. The precursor groups can be changed to a different such group, or to the groups defined in formula I, at any time during the reaction sequence. In the case where X¹ to X⁵ represents a halogen group, such halogen groups may be inter-converted one or more times, after or during the processes described above for the preparation of compounds of formula I. Appropriate reagents include NiCl₂ (for the conversion to a chloro group). In this respect, the skilled person may also refer to “Comprehensive Organic Functional Group Transformations” by A. R. Katritzky, O. Meth-Cohn and C. W. Rees, Pergamon Press, 1995.

Other transformations that may be mentioned include the conversion of a halo group (preferably iodo or bromo) to a cyano or 1-alkynyl group (e.g. by reaction with a compound which is a source of cyano anions (e.g. sodium, potassium, copper (I) or zinc cyanide) or with a 1-alkyne, as appropriate). The latter reaction may be performed in the presence of a suitable coupling catalyst (e.g. a palladium and/or a copper based catalyst) and a suitable base (e.g. a tri-(C₁₋₆ alkyl)amine such as triethylamine, tributylamine or ethyldiisopropylamine). Further, amino groups and hydroxy groups may be introduced in accordance with standard conditions using reagents known to those skilled in the art.

Compounds of the invention may be isolated from their reaction mixtures using conventional techniques.

It will be appreciated by those skilled in the art that, in the processes described above and hereinafter, the functional groups of intermediate compounds may need to be protected by protecting groups. For example the pyrazole nitrogen may need to be protected. Suitable nitrogen-protecting groups include those which form:

(i) carbamate groups (i.e. alkoxy- or aryloxy-carbonyl groups);
(ii) amide groups (e.g. acetyl groups);
(iii) N-alkyl groups (e.g. benzyl or SEM (i.e. a —CH₂OC₂H₄Si(CH₃)₃ group) groups);
(iv) NV-sulfonyl groups (e.g. N-arylsulfonyl groups);
(v) N-phosphinyl and N-phosphoryl groups (e.g. diarylphosphinyl and diarylphosphoryl groups); or
(vi) N-silyl group (e.g. a N-trimethylsilyl group).

Further protecting groups for the pyrazole nitrogen include a methyl group, which methyl group may be deprotected under standard conditions, such as employing a pyridine hydrochloride salt at elevated temperature, for example using microwave irradiation in a sealed vessel at 200° C.

The protection and deprotection of functional groups may take place before or after a reaction in the above-mentioned schemes.

Protecting groups may be removed in accordance with techniques that are well known to those skilled in the art and as described hereinafter. For example, protected compounds/intermediates described herein may be converted chemically to unprotected compounds using standard deprotection techniques.

The type of chemistry involved will dictate the need, and type, of protecting groups as well as the sequence for accomplishing the synthesis.

The use of protecting groups is fully described in “Protective Groups in Organic Chemistry”, edited by J W F McOmie, Plenum Press (1973), and “Protective Groups in Organic Synthesis”, 3rd edition, T. W. Greene & P. G. M. Wutz, Wiley-Interscience (1999).

### Medical and Pharmaceutical Uses

Compounds of the invention are useful because they possess pharmacological activity. Such compounds are therefore indicated as pharmaceuticals. According to a further aspect of the invention there is provided a compound of formula I, as hereinbefore defined but without provisos (a) to (d) and (ID, or a pharmaceutically-acceptable salt thereof, for use as a pharmaceutical and/or in isolated (i.e. ex vivo) form.

Although compounds of the invention may possess pharmacological activity as such, certain pharmaceutically-acceptable (e.g. “protected”) derivatives of compounds of the invention may exist or be prepared which may not possess such activity, but may be administered parenterally or orally and thereafter be metabolised in the body to form compounds of the invention. Such compounds (which may possess some pharmacological activity, provided that such activity is appreciably lower than that of the “active” compounds to which they are metabolised), may therefore be described as “prodrugs” of compounds of the invention. All prodrugs of compounds of the invention are included within the scope of the invention.

By “prodrug of a compound of the invention”, we include compounds that form a compound of the invention, in an experimentally-detectable amount, within a predetermined time (e.g. about 1 hour), following oral or parenteral administration.

Compounds of the invention are useful because, in particular, they may inhibit the activity of lipoxygenases (and particularly 15-lipoxygenase), i.e. they prevent the action of 15-lipoxygenase or a complex of which the 15-lipoxygenase enzyme forms apart and/or may elicit a 15-lipoxygenase modulating effect, for example as may be demonstrated in the test described below. Compounds of the invention may thus be useful in the treatment of those conditions in which inhibition of a lipoxygenase, and particularly 15-lipoxygenase, is required.

Compounds of the invention are thus expected to be useful in the treatment of inflammation.

The term “inflammation” will be understood by those skilled in the art to include any condition characterised by a localised or a systemic protective response, which may be elicited by physical trauma, infection, chronic diseases, such as those mentioned hereinbefore, and/or chemical and/or physiological reactions to external stimuli (e.g. as part of an allergic response). Any such response, which may serve to destroy, dilute or sequester both the injurious agent and the injured tissue, may be manifest by, for example, heat, swelling, pain, redness, dilation of blood vessels and/or increased blood flow, invasion of the affected area by white blood cells, loss of function and/or any other symptoms known to be associated with inflammatory conditions.

The term “inflammation” will thus also be understood to include any inflammatory disease, disorder or condition per se, any condition that has an inflammatory component associated with it, and/or any condition characterised by inflammation as a symptom, including inter alia acute, chronic, ulcerative, specific, allergic and necrotic inflammation, and other forms of inflammation known to those skilled in the art. The term thus also includes, for the purposes of this invention, inflammatory pain and/or fever.

Accordingly, compounds of the invention may be useful in the treatment of asthma, chronic obstructive pulmonary disease (COPD), pulmonary fibrosis, allergic disorders, rhinitis, inflammatory bowel disease, ulcers, inflammatory pain, fever, atherosclerosis, coronary artery disease, vasculitis, pancreatitis, arthritis, osteoarthritis, rheumatoid arthritis, conjunctivitis, iritis, scleritis, uveitis, wound healing, dermatitis, eczema, psoriasis, stroke, diabetes, autoimmune diseases, Alzheimer's disease, multiple sclerosis, sarcoidosis, Hodgkin's disease and other malignancies, and any other disease with an inflammatory component.

Compounds of the invention may also have effects that are not linked to inflammatory mechanisms, such as in the reduction of bone loss in a subject Conditions that may be mentioned in this regard include osteoporosis, osteoarthritis, Paget's disease and/or periodontal diseases. Compounds of formula I and pharmaceutically acceptable salts thereof may thus also be useful in increasing bone mineral density, as well as the reduction in incidence and/or healing of fractures, in subjects.

Compounds of the invention are indicated both in the therapeutic and/or prophylactic treatment of the above-mentioned conditions.

According to a further aspect of the present invention, there is provided a method of treatment of a disease which is associated with, and/or which can be modulated by inhibition of, a lipoxygenase (such as 15-lipoxygenase), and/or a method of treatment of a disease in which inhibition of the activity of a lipoxygenase, and particularly 15-lipoxygenase, is desired and/or required (e.g. inflammation), which method comprises administration of a therapeutically effective amount of a compound of formula I as hereinbefore defined but without the provisos, or a, pharmaceutically-acceptable salt thereof, to a patient suffering from, or susceptible to, such a condition.

“Patients” include mammalian (including human) patients.

The term “effective amount” refers to an amount of a compound, which confers a therapeutic effect on the treated patient. The effect may be objective (i.e. measurable by some test or marker) or subjective (i.e. the subject gives an indication of or feels an effect).

Compounds of the invention will normally be administered orally, intravenously, subcutaneously, buccally, rectally, dermally, nasally, tracheally, bronchially, sublingually, by any other parenteral route or via inhalation, in a pharmaceutically acceptable dosage form.

Compounds of the invention may be administered alone, but are preferably administered by way of known pharmaceutical formulations, including tablets, capsules or elixirs for oral administration, suppositories for rectal administration, sterile solutions or suspensions for parenteral or intramuscular administration, and the like.

Such formulations may be prepared in accordance with standard and/or accepted pharmaceutical practice.

According to a further aspect of the invention there is thus provided a pharmaceutical formulation including a compound of formula I, as hereinbefore defined but without provisos (a) to (d) and (f), or a pharmaceutically-acceptable salt thereof, in admixture with a pharmaceutically acceptable adjuvant, diluent or carrier.

The invention further provides a process for the preparation of a pharmaceutical formulation, as hereinbefore defined, which process comprises bringing into association a compound of formula I, as hereinbefore defined but without provisos (a) to (d) and (f, or a pharmaceutically acceptable salt thereof with a pharmaceutically-acceptable adjuvant, diluent or carrier.

Compounds of the invention may also be combined with other therapeutic agents that are useful in the treatment of inflammation as defined herein (e.g. NSAIDs, coxibs, corticosteroids, analgesics, inhibitors of 5-lipoxygenase, inhibitors of FLAP (5-lipoxygenase activating protein), and leukotriene receptor antagonists (LTRas), and/or other therapeutic agents that are useful in the treatment of inflammation).

According to a further aspect of the invention, there is provided a combination product comprising:
(A) a compound of formula I, as hereinbefore defined but without the provisos, or a pharmaceutically-acceptable salt thereof; and
(B) another therapeutic agent that is useful in the treatment of inflammation,
   wherein each of components (A) and (B) is formulated in admixture with a pharmaceutically-acceptable adjuvant, diluent or carrier.

Such combination products provide for the administration of compound of the invention in conjunction with the other therapeutic agent, and may thus be presented either as separate formulations, wherein at least one of those formulations comprises compound of the invention and at least one comprises the other therapeutic agent, or may be presented (i.e. formulated) as a combined preparation (i.e. presented as a single formulation including compound of the invention and the other therapeutic agent).

Thus, there is further provided:

(1) a pharmaceutical formulation including a compound of formula I, as hereinbefore defined but without the provisos, or a pharmaceutically-acceptable salt thereof, another therapeutic agent that is useful in the treatment of inflammation, and a pharmaceutically-acceptable adjuvant, diluent or carrier; and
(2) a kit of parts comprising components:
(a) a pharmaceutical formulation including a compound of formula I, as hereinbefore defined but without the provisos, or a pharmaceutically-acceptable salt thereof, in admixture with a pharmaceutically-acceptable adjuvant, diluent or carrier, and
(b) a pharmaceutical formulation including another therapeutic agent that is useful in the treatment of inflammation in admixture with a pharmaceutically-acceptable adjuvant, diluent or carrier, which components (a) and (b) are each provided in a form that is suitable for administration in conjunction with the other.

The invention further provides a process for the preparation of a combination product as hereinbefore defined, which process comprises bringing into association a compound of formula I, as hereinbefore defined but without the provisos, or a pharmaceutically acceptable salt thereof with the other therapeutic agent that is useful in the treatment of inflammation, and at least one pharmaceutically-acceptable adjuvant, diluent or carrier.

By “bringing into association”, we mean that the two components are rendered suitable for administration in conjunction with each other.

Thus, in relation to the process for the preparation of a kit of parts as hereinbefore defined, by bringing the two components “into association with” each other, we include that the two components of the kit of parts may be:

(i) provided as separate formulations (i.e. independently of one another), which are subsequently brought together for use in conjunction with each other in combination therapy; or
(ii) packaged and presented together as separate components of a “combination pack” for use in conjunction with each other in combination therapy.

Compounds of the invention may be administered at varying doses. Oral, pulmonary and topical dosages may range from between about 0.01 mg/kg of body weight per day (mg/kg/day) to about 100 mg/kg/day, preferably about 0.01 to about 10 mg/kg/day, and more preferably about 0.1 to about 5.0 mg/kg/day. For e.g. oral administration, the compositions typically contain between about 0.01 mg to about 500 mg, and preferably between about 1 mg to about 100 mg, of the active ingredient. Intravenously, preferred doses will range from about 0.001 to about 10 mg/kg/hour during constant rate infusion. Advantageously, compounds may be administered in a single daily dose, or the total daily dosage may be administered in divided doses of two, three or four times daily.

In any event, the physician, or the skilled person, will be able to determine the actual dosage which will be most suitable for an individual patient, which is likely to vary with the route of administration, the type and severity of the condition that is to be treated, as well as the species, age, weight, sex, renal function, hepatic function and response of the particular patient to be treated. The above-mentioned dosages are exemplary of the average case; there can, of course, be individual instances where higher or lower dosage ranges are merited, and such are within the scope of this invention.

Compounds of the invention may have the advantage that they are effective and/or selective inhibitors of lipoxygenases, and particularly 15-lipoxygenase.

Compounds of the invention may also have the advantage that they may be more efficacious than, be less toxic than, be longer acting than, be more potent than, produce fewer side effects than, be more easily absorbed than, and/or have a better pharmacokinetic profile (e.g. higher oral bioavailability and/or lower clearance) than, and/or have other useful pharmacological physical, or chemical properties over, compounds known in the prior art, whether for use in the stated indications or otherwise.

### Biological Test

The assay employed takes advantage of the ability of lipoxygenases to oxidize polyunsaturated fatty acids, containing a 1,4-cis-pentadiene configuration, to their corresponding hydroperoxy or hydroxyl derivatives. In this particular assay, the lipoxygenase was a purified human 15-lipoxygenase and the fatty acid was arachidonic acid. The assay is performed at room temperature (20-22° C.) and the following are added to each well in a 96-well microtiter plate:

a) 35 μL phosphate buffered saline (PBS) (pH 7.4);
b) inhibitor (i.e. compound) or vehicle (0.5 μl DMSO);
c) 10 μL of a 10× concentrated solution of 15-lipoxygenase in PBS. The plates are incubated for 5 minutes at room temperature;
d) 5 μl of 0.125 mM arachidonic acid in PBS. The plate is then incubated for 10 minutes at room temperature;
e) the enzymatic reaction is terminated by the addition of 100 μl methanol; and

f) the amount of 15-hydroperoxy-eicosatetraenoic acid or 15-hydroxy-eicosatetraenoic acid is measured by reverse phase HPLC.

The invention is illustrated by way of the following examples, in which the following abbreviations may be employed:
DMAP 4-dimethylaminopyridine
DMF dimethylformamide
DIPEA diisopropylethylamine
ETOAc ethyl acetate
Et₂O diethyl ether
MeOH methanol
MS mass spectrum
NMR nuclear magnetic resonance
rt room temperature
TBTU O-benzotriazol-1-yl-N,N,N′,N′-tetramethyluronium tetrafluoroborate
LC liquid chromatography

Starting materials and chemical reagents specified in the syntheses described below are commercially available from, e.g. Sigma-Aldrich Fine Chemicals.

Unless otherwise stated, one or more tautomeric forms of compounds of the examples described hereinafter may be prepared in situ and/or isolated. All tautomeric forms of compounds of the examples described hereinafter should be considered to be disclosed.

### Synthesis Of Intermediates:

Dipyrazolo[1.5-a;1′,5′-d]pyrazine-4,9-dione (I)

The intermediate was prepared in accordance with the procedure described in Example 6, step (a) of international patent application WO 2004/080999.

### EXAMPLES

### Example 1

### N-(2-Chloro-4-fluorophenyl)pyrazole-3-carboxamide

The title compound was prepared in accordance with the procedure described in Example 48 of international patent application WO 2004/080999.

### Example 2

### N-(2-Bromo-4-trifluoromethoxyphenyl)pyrazole-3-carboxamide

The title compound was prepared in accordance with the procedure described in Example 68 of international patent application WO 2004/080999.

### Example 3

### N-(2-Methoxy-5-trifluoromethylphenyl)pyrazole-3-carboxamide

The title compound was prepared analogously to the procedure described in Example 1 above.

### ¹H NMR (DMSO-d₆) δ 13.55 (br s, 1H), 9.49 (s, 1H), 8.67 (s, 1H), 7.94 (d, 1H), 7.47 (dd, 1H), 7.29 (d, 1H), 6.81 (d, 1H), 3.99 (s, 3H).

### Example 4

### N-(2-Fluoro-5-trifluoromethylphenyl)pyrazole-3-carboxamide

The title compound was prepared in accordance with the procedures described in Example 60 of international patent application WO 2004/080999.

### ¹H NMR (DMSO-d₆) δ 13.58 (br s, 1H), 10.02 (br s, 1H), 8.26 (dd, 1H), 7.91 (d, 1H), 7.65-7.50 (m, 2M), 6.83 (d, 1H).

### Example 5

### N-(4-Fluoro-2-trifluoromethylphenyl)pyrazole-3-carboxamide

Dipyrazolo[1,5-a;1′,5′-d]pyrazine-4,9-dione (1.00 g, 5.32 mmol; intermediate (I) above), 4-fluoro-2-trifluoromethylaniline (2.382 g, 13.3 mmol) and DMAP (0.650 g, 5.32 mmol) were dissolved in chloroform (3 mL) and the mixture was then heated at 80° C. for 18 h in a sealed vessel. The reaction was then cooled to ambient temperature and ethanol (20 mL) was added to the reaction mixture. The reaction mixture was then filtered to remove the solids, the filtrate was then concentrated under reduced pressure and the residue crystallised from MeOH to afford the title compound, 290 mg (10%).

MS (M⁺+H) m/z=274

### Example 6

### N-(2,4-Difluorophenyl)pyrazole-3-carboxamide

A mixture of dipyrazolo[1,5-a;1′,5′-d]pyrazine-4,9-dione (160 mg, 0.531 mmol; intermediate (I) above), 2,4-difluoroaniline (343 mg, 2.66 mmol), DMAP (65 mg, 0.53 mmol) and DMF (5 mL) were heated at 140° C. overnight. The mixture was then cooled and concentrated in vacuo and the solid residue purified by flash column chromatography (EtOAc). After further purification on preparative LC, the title product was obtained as a white solid, 55 mg (46%).

MS (M⁺+H) m/z=224

### ¹H—MR(CD₃OD), δ 8.12-7.90 (m, 1H), 7.85-7.70 (m, 1H), 7.20-6.95 (m, 2H), 6.92-6.80 (m, 1H).

### Example 7

### N-(2,3,4-Trifluorophenyl)pyrazole-3-carboxamide

A mixture of dipyrazolo[1,5-a;1′,5′-d]pyrazine-4,9-dione (511 mg, 2.72 mmol; intermediate (I) above), 2,3,4-trifluoroaniline (211 mg, 1.43 mmol), DMAP (166 mg, 1.36 mmol) and DMF (5 mL) were heated at 140° C. for 2 h. The mixture was cooled and concentrated under reduced pressure and the solid residue was then purified by flash column chromatography (Et₂O). Recrystallisation from MeOH/water furnished the title compound as white crystals, 153 mg (44%).

MS (M⁺+H) m/z=242

### ¹H—NMR (DMSO-d₆), δ 9.93 (s, 1H), 7.86 (s, 1H), 7.50-7.20 (m, 2H), 6.73 (s, 1H).

### Example 8

### N-(2,4-Dichlorophenyl)pyrazole-3-carboxamide

TBTU (515 mg, 1.6 mmol) was added to a solution of pyrazole-3-carboxylic acid (150 mg, 1.3 mmol), 2,4-dichloroaniline (1.0 g, 7.3 mmol) and DIPEA (340 mg, 2.6 mmol) in DMF (10 mL). The mixture was stirred at rt overnight followed by addition of water (50 mL) and extraction with Et₂O (3×30 mL). The combined organic phases were washed with water (50 mL), dried (Na₂SO₄) and concentrated in vacuo to furnish a yellow-brown solid that was purified by flash column chromatography (EtOAc/heptane, gradient 1:10 to 1:1) followed by recrystallisation from EtOAc/heptane to furnish the title compounds as colourless crystals, 20 mg (6%).

### ¹H—NMR (DMSO-d₆), δ 9.37 (s, 1H), 8.47 (d, 1H), 7.54 (d, 1H), 7.38 (s, 1H), 7.35 (d, 1H), 7.21 (dd, 1H), 6.81 (d, 1H).

### Example 9

### N-(4-Chloro-2-methylphenyl)pyrazole-3-carboxamide

A mixture of dipyrazolo[1,5-a;1′,5′-d]pyrazine-4,9-dione (192 mg, 1.02 mmol; intermediate (I) above), 4-chloro-o-toluidine (113 mg, 0.80 mmol), DMF (0.7 mL) and pyridine (7 mL) were heated at 80° C. for 24 h. The mixture was allowed to cool and then concentrated under reduced pressure. The residue was purified by flash column chromatography (heptane/EtOAc 60:40) to give the title compound as a white solid, 31 mg (16%).

### ¹H—NMR (DMSO-d₆), δ 13.39 (s, 1H), 9.52 (s, 1H), 7.89 (s, 1H), 7.59 (d, 1H), 7.33 (s, 1H), 7.24 (d, 1H), 6.75 (s, 1H), 2.25 (s, 3H).

### Example 10

### N-(2-Chloro-4-methylphenyl)pyrazole-3-carboxamide

The title compound was prepared in accordance with the procedure described in Example 9 above using 2-chloro-p-toluidine (113 mg, 0.80 mmol) to furnish the title compound as a white solid, 27 mg (14%).

### ¹H—NMR (DMSO-d₆), δ 13.45 (s, 1H), 9.48 (s, 1H), 8.00 (d, 1H), 7.92 (s, 1H), 7.35 (s, 1H), 7.16 (d, 1H), 6.77 (s, 1H), 2.28 (s, 3H).

### Example 11

### N-(5-Chloro-2-cyanophenyl)pyrazole-3-carboxamide

The title compound was prepared in accordance with the procedures described in Example 26 of international patent application WO 2004/080999.

### Example 12

### N-(2.4.5-Trichlorophenyl)pyrazole-3-carboxamide

A mixture of dipyrazolo[1,5-a;1′,5′-d]pyrazine-4,9-dione (188 mg, 1.00 mmol; intermediate (I) above) and 2,4,5-trichloroaniline (158 mg, 0.80 mmol) was suspended in pyridine (2 mL) and DMF (0.5 mL). The mixture was heated at 80° C. for 2 h and a second portion of intermediate (I) (94 mg, 0.50 mmol) was added followed by prolonged heating at 80° C. for 16 h. After cooling, the mixture was concentrated in vacuo and the residue was purified by flash column chromatography (EtOAc/heptane, gradient 1:5 to 1:0) to give 13.6 mg (6%) of the title compound.

### H—NMR (DMSO-d₆), δ 13.86 (br s, 1H), 9.66 (s, 1H), 8.43 (s, 1H), 7.96 (m, 1H), 6.82 (s, 1H).

### Example 13

### N-(2,5-Dichlorophenyl)pyrazole-3-carboxamide

The title compound was prepared in accordance with the procedure described in Example 12 above from dipyrazolo[1,5-a;1′,5′-d]pyrazine-4,9-dione (188 mg, 1.00 mmol; intermediate (I) above) and 2,5-dichloroaniline (130 mg, 0.80 mmol) to furnish 19 mg (9%) of the desired compound.

### ¹H—NMR (DMSO-d₆), δ 13.73 (br s, 1H), 9.63 (br s, 1H), 8.29 (br s, 1H), 7.52 (s, 1H), 7.59 (d, 1H), 7.26 (dd, 1H), 6.83 (s, 1H).

### Example 14

### N-(2,4-Dimethylphenyl)pyrazole-3-carboxamide

The title compound was prepared in accordance with the procedure described in Example 12 from dipyrazolo[1,5-a;1′,5′-d]pyrazine-4,9-dione (188 mg, 1.00 mmol; intermediate (I) above) and 2,4-xylidine (96.9 mg, 0.8 mmol).

### ¹H—NMR (DMSO-d₆), δ 13.37 (s, 1H); 9.37 (s 1H); 7.87 (s, 1H); 7.22 (d, 1H); 7.00 (m, 2H); 6.73 (s, 1H); 2.27 (s, 3H), 2.98 (s, 3H).

### Example 15

### N-(4-Bromo-2-trifluoromethoxyphenyl)pyrazole-3-carboxamide

The title compound was prepared in accordance with the procedure described in Example 9 above using 4-bromo-2-trifluoromethoxyaniline (151 μL, 256 mg, 1.00 mmol) to furnish the title compound as a white solid, 116 mg (33%).

### M.p. 185-187° C.

### ¹H—NMR (DMSO-d₆) δ 13.54 (s, 1H), 9.61 (s, 1H), 8.07 (d, 1H), 7.94 (d, 1H), 7.72 (s, 1H), 7.68 (dd, 1H), 6.81 (d, 1H).

### Example 16

### N-(4-Iodo-2-trifluoromethoxyphenyl)pyrazole-3-carboxamide

A mixture of 4-iodo-2-trifluoromethoxyaniline (152 mg, 0.50 mmol), dipyrazolo[1,5-a;1′,5′-d]pyrazine-4,9-dione (94 mg, 0.50 mmol; intermediate (I) above), DMAP (6.0 mg, 0.05 mmol), pyridine (1.5 mL) and DM (0.5 mL) was stirred at 80° C. for 45 h. The reaction mixture was concentrated in vacuo and the residue was purified by chromatography (EtOAc/heptane) to give the title compound as a white solid, 73 mg (37%).

### M.p. 194.1-194.6° C.

### ¹H—NMR (DMSO-d₆) δ 13.66-13.44 (br s, 1H), 9.65-9.53 (br s, 1H), 8.02-7.84 (r, 2H), 7.84-7.77 (m, 2H), 6.82 (s, 1H).

### Example 17

### N-(4-Chloro-2-cyanophenyl)pyrazole-3-carboxamide

A mixture of 4-chloro-2-cyanophenylaniline (150 mg, 0.98 mmol), dipyrazolo[1,5-a;1′,5′-d]pyrazine-4,9-dione (185 mg, 0.98 mmol; intermediate (I) above), DMAP (120 mg, 0.98 mmol) in DMF (2 mL) was heated in a sealed vial in Biotage® Initiator™ microwave reactor (2.45 GHz, 300 W max. power) at 160° C. for 1 h. The mixture was poured into water (50 mL) and white precipitate formed was filtered off. The crude product was recrystallized from ethanol to give 139 mg (57%) of the title compound.

### MS (M⁺+H) m/z 247.

### ¹H—NMR (DMSO-d₆) δ 13.57 (s, 1H), 10.24 (s, 1H), 8.04 (d, 1H), 7.95 (d, 1H), 7.88 (d, 1H), 7.79 (dd, 1H), 6.82 (d, 1H).

### Example 18

### N-(5-Chloro-2-hydroxyphenyl)pyrazole-3-carboxamide

The title compound was prepared in accordance with the procedure described in Example 17 above using 5-chloro-2-hydroxyaniline (72 mg, 0.50 mmol) to furnish the title compound as a white solid, 86 mg (72%).

### MS (M⁺+H) m/z 238.

### ¹H—NMR (DMSO-d₆) δ 13.46 (s, 1H), 10.75 (s, 1H), 9.39 (s, 1H), 8.32 (s, 1H), 7.93 (s, 1H), 6.96-6.98 (m, 2H), 6.78 (dd, 1H).

### Example 19

Title compounds of the Examples were tested in the biological test described above and were found to exhibit an IC₅₀ of 10 μM or below. For example, the following representative compounds of the examples exhibited the following IC₅₀ values:

### Example 2 160 nM

### Example 7 1800 nM

### Example 9 750 nM

### Example 13 1100 nM

### Example 14 840 nM

### Example 15 140 nM

(f) X¹ represents —CH₃ and X⁴ and X⁵ both represent H, then X³ does not represent —CH₃.
2. A compound of formula I,

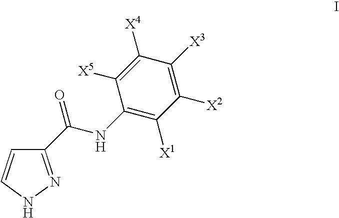

wherein
X¹ represents halo, —CN, —NO₂, —R¹ or —OR²;
X² to X⁵ independently represent H, halo, —CN, —NO₂, —R¹ or —OR², wherein at least one of X³ or X⁴ is other than H;
R² represents H or R¹; and
R¹ represents C₁₋₆ alkyl optionally substituted by one or more substituents selected from F, Cl, —OCH₃, —OCH₂CH₃, —OCHF₂ and —OCF₃,
or a pharmaceutically-acceptable salt thereof, for use as a pharmaceutical, provided that when X² and X³ both represent H, X¹ represents —OH and X⁴ and represents t-Bu, then X⁵ does not represent —OH.
3. A compound as claimed in claim 1 or claim 2, wherein X⁵ represents H.
4. A compound as claimed in any one of the preceding claims, wherein R¹ and/or R² independently represent C₁₋₆ allyl optionally substituted by one or more substituents selected from F, —OCH₃, —OCH₂CH₃, —OCHF₂ and —OCF₃.
5. A compound as claimed in claim 4, wherein R¹ and/or R² independently represent C₁₋₄ alkyl optionally substituted as defined in claim 4.

1. A compound of formula I, 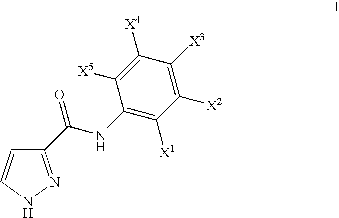
wherein
X¹ represents halo, —CN, —NO₂, —R¹ or —OR²;
X² to X⁵ independently represent H, halo, —CN, —NO₂, —R¹ or —OR², wherein at least one of X³ or
X⁴ is other than H;
R² represents H or R¹; and
R¹ represents C₁₋₆ alkyl optionally substituted by one or more substituents selected from F, Cl, —OCH₃, —OCH₂CH₃, —OCHF₂ and —OCF₃;
or a pharmaceutically acceptable salt thereof;
provided that when X² represents H:
(a) X¹ represents —CN and X³ and X⁵ both represent H, then X⁴ does not represent Cl;
(b) X¹ represents Cl and X⁴ and X⁵ both represent H, then X³ does not represent F;
(c) X¹ represents F and X³ and X⁵ both represent H, then X⁴ does not represent —CF₃;
(d) X¹ represents Br and X⁴ and X⁵ both represent H, then X³ does not represent —OCF₃;
(e) X¹ represents —OH and X³ represents H and X⁴ and represents t-Bu, then X⁵ does not represent —OH; and
(f) X¹ represents —CH₃ and X⁴ and X⁵ both represent H, then X³ does not represent —CH₃.

2. A compound of formula I, 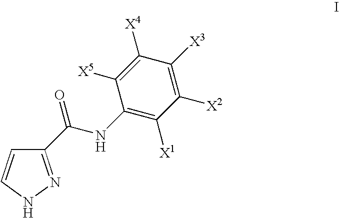
wherein
X¹ represents halo, —CN, —NO₂, —R¹ or —OR²;
X² to X⁵ independently represent H, halo, —CN, —NO₂, —R¹ or —OR², wherein at least one of X³ or
X⁴ is other than H;
R² represents H or R¹; and
R¹ represents C₁₋₆ alkyl optionally substituted by one or more substituents selected from F, Cl, —OCH₃, —OCH₂CH₃, —OCHF₂ and —OCF₃,
or a pharmaceutically-acceptable salt thereof, for use as a pharmaceutical,
provided that when X² and X³ both represent H, X¹ represents —OH and X⁴ and represents t-Bu, then X⁵ does not represent —OH.

3. A compound as claimed in claim 1 or claim 2, wherein X⁵ represents H.

4. A compound as claimed in claim 2 wherein R¹ and/or R² independently represent C₁₋₆ alkyl optionally substituted by one or more substituents selected from F, —OCH₃, —OCH₂CH₃, —OCHF₂ and —OCF₃.

5. A compound as claimed in claim 4, wherein R¹ and/or R² independently represent C₁₋₄ alkyl optionally substituted as defined in claim 4.

6. A compound as claimed in claim 2, wherein X¹ represents Br, Cl, F, —CN, R¹ or —OR².

7. A compound as claimed in claim 2, wherein X² to X⁴ represents 1, H, F, Cl, Br, R¹ or —OR².

8. A compound as claimed in claim 7, wherein X² to X⁴ represents H, F, Cl, Br, R¹ or —OR².

9. A compound as claimed in claim 2, wherein X³ represents Br, I, H, Cl, F, R¹ or —OR².

10. A compound as claimed in claim 9, wherein X³ represents H, Cl, F, R¹ or OR².

11. A compound as claimed in claim 2, wherein X⁴ represents H, Cl, F or R¹.

12. A compound as claimed in claim 2, wherein X² represents H, Cl or F.

13. A compound as claimed in claim 2, wherein R¹ and/or R² independently represent H or C₁₋₂ alkyl optionally substituted by one or more F substituent.

14. A compound as claimed in claim 2, wherein R¹ and/or R² independently represent C₁₋₂ alkyl optionally substituted by one or more F substituent.

15. A compound as claimed in claim 13, wherein R¹ and/or R² independently represent H, methyl, ethyl, difluoromethyl, trifluoromethyl or 1,1,1-trifluoroethyl.

16. A compound as claimed in claim 14, wherein R¹ and/or R² independently represent methyl, ethyl, difluoromethyl, trifluoromethyl or 1,1,1-trifluoroethyl.

17. A pharmaceutical formulation including a compound of formula I, as defined in claim 2, or a pharmaceutically-acceptable salt thereof, in admixture with a pharmaceutically acceptable adjuvant, diluent or carrier.

18. (canceled)

19. Method as claimed in claim 22, wherein the lipoxygenase is 15-lipoxygenase.

20. Method as claimed in claim 19, wherein the disease is inflammation and/or has an inflammatory component.

21. Method as claimed in claim 20, wherein the inflammatory disease is asthma, chronic obstructive pulmonary disease (COPD), pulmonary fibrosis, an allergic disorder, rhinitis, inflammatory bowel disease, an ulcer, inflammatory pain, fever, atherosclerosis, coronary artery disease, vasculitis, pancreatitis, arthritis, osteoarthritis, rheumatoid arthritis, conjunctivitis, iritis, scleritis, uveitis, a wound, dermatitis, eczema, psoriasis, stroke, diabetes, autoimmune diseases, Alzheimer's disease, multiple sclerosis, sarcoidosis, Hodgkin's disease or another malignancy.

22. A method of treatment of a disease in which inhibition of the activity of a lipoxygenase is desired and/or required, which method comprises administration of a therapeutically effective amount of a compound of formula I as defined in claim 1 or claim 2 but without the provisos, or a pharmaceutically-acceptable salt thereof, to a patient suffering from, or susceptible to, such a condition.

23. A combination product comprising:
(A) a compound of formula I as defined in claim 1 or claim 2 but without the provisos, or a pharmaceutically-acceptable salt thereof; and
(B) another therapeutic agent that is useful in the treatment of inflammation, wherein each of components (A) and (B) is formulated in admixture with a pharmaceutically-acceptable adjuvant, diluent or carrier.

24. A combination product as claimed in claim 23 which comprises a pharmaceutical formulation including a compound of formula I as defined in claim 1 or claim 2 but without the provisos, or a pharmaceutically-acceptable salt thereof, another therapeutic agent that is useful in the treatment of inflammation, and a pharmaceutically-acceptable adjuvant, diluent or carrier.

25. A combination product as claimed in claim 23 which comprises a kit of parts comprising components:
(a) a pharmaceutical formulation including a compound of formula I as defined in claim 1 or claim 2 but without the provisos, or a pharmaceutically-acceptable salt thereof, in admixture with a pharmaceutically-acceptable adjuvant, diluent or carrier; and
(b) a pharmaceutical formulation including another therapeutic agent that is useful in the treatment of inflammation in admixture with a pharmaceutically-acceptable adjuvant, diluent or carrier,
which components (a) and (b) are each provided in a form that is suitable for administration in conjunction with the other.

26. A process for the preparation of a compound of formula I as defined in claim 1, which comprises:
(i) reaction of pyrazole-3-carboxylic acid, or a N-protected and/or O-protected derivative thereof, with a compound of formula II, 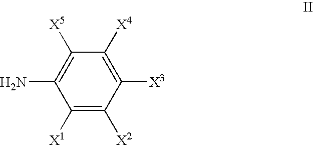
wherein X¹ to X⁵ are as defined in claim 1;
(ii) reaction of pyrazole, or a N-protected derivative thereof, with a suitable base, followed by reaction with a compound of formula III, 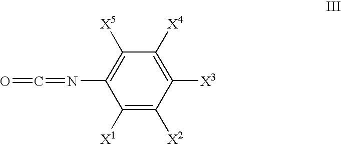
wherein X¹ to X⁵ are as defined in claim 1, followed by quenching with a suitable proton source;
(iii) reaction of dipyrazolo[1,5-a;1′,5′-d]pyrazine-4,9-dione, with a compound of formula II as defined above; or
(iv) reaction of pyrazole-3-carboxamide, or a N-protected derivative thereof, with a compound of formula IV, 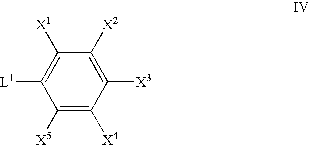
wherein L¹ represents a suitable leaving group and X¹ to X⁵ are as defined in claim 1.

27. A process for the preparation of a pharmaceutical formulation as defined in claim 17, which process comprises bringing into association a compound of formula I, as defined in claim 2 or a pharmaceutically acceptable salt thereof with a pharmaceutically-acceptable adjuvant, diluent or carrier.

28. A process for the preparation of a combination product as defined in claim 23, which process comprises bringing into association a compound of formula I, as defined in claim 1 but without the provisos, or a pharmaceutically acceptable salt thereof with the other therapeutic agent that is useful in the treatment of inflammation, and at least one pharmaceutically-acceptable adjuvant, diluent or carrier.
